(19) United States
(12) Patent Application Publication
(10) Pub. No.: US 2024/0174716 A1
(43) Pub. Date: May 30, 2024

(54) TRANSPORTER PEPTIDES AND APPLICATION THEREOF

(71) Applicant: Vacino Biotech Co. Ltd, Taipei (JP)

(72) Inventors: Jia-Ming CHANG, Taipei (JP); Yi-RU LEE, Taipei (JP)

(21) Appl. No.: 18/502,348

(22) Filed: Nov. 6, 2023

Related U.S. Application Data
(60) Provisional application No. 63/385,266, filed on Nov. 29, 2022.

(51) Int. Cl. C07K 7/06; A61K 47/64; C12N 15/86

(52) U.S. Cl. CPC C07K 7/06; A61K 47/644; C12N 15/86; A61K 38/00; C12N 2750/14143

(57) ABSTRACT

The present invention relates to transporter peptides that can bind to a transferrin receptor (TfR). The transporter peptides can be conjugated covalently or noncovalently to an effector agent to form a transporter peptide conjugate, or a nucleic acid encoding the transporter peptides and an effector agent is expressed to form a recombinant transporter peptide conjugate. The transporter peptide conjugate and the recombinant transporter peptide conjugate deliver the effector agent to a target by binding to a TfR. Binding of the transporter peptides to transferrin receptors on cells of a tissue barrier induces transcytosis of the cells, thereby transporting the transporter peptide conjugate across the tissue barrier. The transporter peptides can serve as a drug delivery system and used for treatment of central nervous system (CNS) diseases.

### CROSS-REFERENCE TO RELATED APPLICATIONS

This application claims priority to and the benefit of U.S. Provisional Application No. 63/385,266, filed on Nov. 29, 2022, the disclosure of which is incorporated by reference in its entirety.

### SUBMISSION OF SEQUENCE LISTING AS ASCII TEXT FILE

This application includes an electronically submitted sequence listing in XML format. The XML file contains a sequence listing entitled “P23-0221US_Sequence_Listing.xml” which was created on Nov. 6, 2023 and is 30,912 bytes in size. The sequence listing contained in this XML file is part of the specification and is hereby incorporated by reference herein in its entirety.

### BACKGROUND OF THE INVENTION

### 1. Field of the Invention

The present invention relates to transporter peptides capable of crossing tissue barriers and methods of using these transporter peptides for delivering effector agents across tissue barriers.

### 2. Description of the Prior Art

The main function of tissue barriers is to restrict the passage of substances. They exist in various tissues and organs in animals and human beings to maintain the environmental stability of a specific area in the body or to protect important organs from the influence of external substances. Examples of tissue barriers are, but not limited to, blood-brain barrier (BBB) and gastrointestinal mucosal barrier, etc.

Blood-brain barrier is located at the central nervous system and is composed of cerebral microvascular endothelial cells (BMECs). Its main function is to protect the brain from external substances. It also controls the passage of specific substances (such as oxygen and nutrients) to ensure normal brain function. However, in clinical applications, the existence of the blood-brain barrier greatly limits the entry of therapeutic drugs into the brain. Relevant clinical studies have pointed out that the proportion of neurological drugs in the brain reaching the brain through the blood-brain barrier is less than 0.1%. Therefore, drug delivery barriers caused by the blood-brain barrier have been a long-standing problem in the fields related to the treatment of central nervous system (CNS) diseases.

### SUMMARY OF THE INVENTION

The present invention is based, at least in part, on the discovery that transporter peptides comprising the amino acid sequence of SEQ ID NO: 1 have the binding affinity to transferrin receptors (TfRs). These transporter peptides can effectively bind to TfRs on the cells of tissue barriers and induce transcytosis of the cells to allow the transporter peptides cross the tissue barriers. In addition, the transporter peptides can effectively bind to the TfRs on a target cells. In some embodiments, the transporter peptides of the present invention comprise, but are not limited to, any one of the amino acid sequences of SEQ ID NO: 2 to SEQ ID NO: 30 and variant sequences at least 70% homologous to one of SEQ ID NO: 2 to SEQ ID NO: 30 and being able to bind to the transferrin receptor.

In some embodiments, the transporter peptides of the present invention further conjugate covalently or non-covalently to an effector agent to form a transporter peptide conjugate. In some embodiments, a recombinant transporter peptide conjugate is formed by expressing a nucleic acid encoding the transporter peptides of the present invention and an effector agent. These transporter peptides transport the effector agent to a target by binding to a TfR. Therefore, the transporter peptides disclosed herein can serve as a drug delivery system. In some embodiments, binding of the transporter peptides to TfRs on a cell of a tissue barrier induces transcytosis of the cell, thereby transport the transporter peptides and the effector agents across the tissue barrier. With the transporter peptides and the transporting methods of the present invention, an effector agent can effectively cross a tissue barrier, especially BBB, and reach the brain. Therefore, the transporter peptides and the transporting methods of the present invention can be broadly used to prevent and/or treat CNS diseases.

Those skilled in the art will recognize, or be able to ascertain using no more than routine experimentation, many equivalents to the specific embodiments of the invention described herein. Such equivalents are intended to be encompassed by the following embodiments.

Embodiment 1. A transporter peptide, consisting of 10 amino acids having the general sequence of

X₁-X₂-X₃-X₄-X₅-X₆-X₇-X₈-X₉-X₁₀

wherein,
X₁ is an acidic amino acid or a polar uncharged amino acid;
X₂ is a nonpolar amino acid;
X₃ is an arbitrary amino acid;
X₄ is an acidic amino acid or a nonpolar amino acid;
X₅ is a nonpolar amino acid;
X₆ is an arbitrary amino acid;
X₇ is a basic amino acid;
X₈ is an acidic amino acid or a basic amino acid;
X₉ is a nonpolar amino acid or a polar uncharged amino acid; and
X₁₀ is an acidic amino acid or a polar uncharged amino acid.

Embodiment 2. A transporter peptide, consisting of 10 amino acids having the general sequence of

X₁-X₂-X₃-X₄-X₅-X₆-X₇-X₈-X₉-X₁₀

wherein,
X₁ is Asp, Glu, Asn, Gln, Cys, Ser, Thr, or Tyr;
X₂ is Ile, Gly, Ala, Leu, Val, Pro, Phe, Trp, or Met;
X₃ is Gly, Ala, Val, Leu, Ile, Phe, Tyr, Trp, His, Asp, Asn, Glu, Gln, Lys, Arg, Ser, Thr, Met, Cys, or Pro;
X₄ is Asp, Glu, Ile, Gly, Ala, Leu, Val, Pro, Phe, Trp, or Met;
X₅ is Ile, Gly, Ala, Leu, Val, Pro, Phe, Trp, or Met;
X₆ is Gly, Ala, Val, Leu, Ile, Phe, Tyr, Trp, His, Asp, Asn, Glu, Gln, Lys, Arg, Ser,
Thr, Met, Cys, or Pro;
X₇ is Lys, His, or Arg;
X₈ is Asp, Glu, Lys, His, or Arg;
X₉ is Ile, Gly, Ala, Leu, Val, Pro, Phe, Trp, Met, Asn, Gln, Cys, Ser, Thr, or Tyr; and
X₁₀ is Asp, Glu, Asn, Gln, Cys, Ser, Thr, or Tyr.

Embodiment 3. A transporter peptide, consisting of 10 amino acids having the amino acid sequence of SEQ ID NO: 1:

(SEQ ID NO: 1)
X₁-Ile-X₃-Val-Leu-X₆-Lys-X₈-X₉-X₁₀,

wherein
X₁ is Asp, Glu, Asn, Gln, Cys, Ser, Thr, or Tyr;
the Ile at the second amino acid site of SEQ ID NO: 1 can be replaced by Gly, Ala, Leu, Val, Pro, Phe, Trp, or Met;
X₃ is Gly, Ala, Val, Leu, Ile, Phe, Tyr, Trp, His, Asp, Asn, Glu, Gln, Lys, Arg, Ser, Thr, Met, Cys, or Pro;
the Val at the fourth amino acid site of SEQ ID NO: 1 can be replaced by Asp, Glu, Ile, Gly, Ala, Leu, Pro, Phe, Trp, or Met;
the Leu at the fifth amino acid site of SEQ ID NO: 1 can be replaced by Ile, Gly, Ala, Val, Pro, Phe, Trp, or Met;
X₆ is Gly, Ala, Val, Leu, Ile, Phe, Tyr, Trp, His, Asp, Asn, Glu, Gln, Lys, Arg, Ser, Thr, Met, Cys, or Pro;
the Lys at the seventh amino acid site of SEQ ID NO: 1 can be replaced by His or Arg;
X₈ is Asp, Glu, Lys, His, or Arg;
X₉ is Ile, Gly, Ala, Leu, Val, Pro, Phe, Trp, Met, Asn, Gln, Cys, Ser, Thr, or Tyr; and
X₁₀ is Asp, Glu, Asn, Gln, Cys, Ser, Thr, or Tyr.

Embodiment 4. The transporter peptide of any one of Embodiments 1 to 3, wherein the transporter peptide is able to bind to a transferrin receptor (TfR).

Embodiment 5. The transporter peptide of any one of Embodiments 1 to 4, wherein the sequence of the transporter peptide is selected from the group consisting of SEQ ID NO: 2 to SEQ ID NO: 30 and variant sequences at least 70% homologous to one of SEQ ID NO: 2 to SEQ ID NO: 30 and being able to bind to the transferrin receptor.

Embodiment 6. The transporter peptide of Embodiment 5, wherein the variant sequences are 70% homologous to one of SEQ ID NO: 2 to SEQ ID NO: 30.

Embodiment 7. The transporter peptide of Embodiment 5, wherein the variant sequences are 80% homologous to one of SEQ ID NO: 2 to SEQ ID NO: 30.

Embodiment 8. The transporter peptide of Embodiment 5, wherein the variant sequences are 90% homologous to one of SEQ ID NO: 2 to SEQ ID NO: 30.

Embodiment 9. A nucleic acid encoding the transporter peptide of any one of Embodiments 1 to 8.

Embodiment 10. The nucleic acid of Embodiment 9, wherein the nucleic acid is deoxyribonucleic acid (DNA).

Embodiment 11. The nucleic acid of Embodiment 9, wherein the nucleic acid is ribonucleic acid (RNA).

Embodiment 12. A vector, comprising the nucleic acid of any one of Embodiments 9 to 11.

Embodiment 13. The vector of Embodiment 12, wherein the vector is an adeno-associated virus (AAV) vector.

Embodiment 14. The vector of Embodiment 12, further comprising a nucleic acid encoding an effector agent.

Embodiment 15. The vector of Embodiment 14, wherein the effector agent is at least one selected from the group consisting of peptides, proteins, antibodies, viral particles, liposomes, endosomes, exosomes, ligands, eukaryotic cells, prokaryotic cells, and microspheres.

Embodiment 16. A recombinant transporter peptide conjugate, which is expressed by the vector of Embodiments 14 or 15.

Embodiment 17. A recombinant host cell, comprising one component selected from the group consisting of the transporter peptide of any one of Embodiments 1 to 8, the nucleic acid of any one of Embodiments 9 to 11, and the vector of any one of Embodiments 12 to 15.

Embodiment 18. A transporter peptide conjugate, consisting of the transporter peptide of any one of Embodiments 1 to 8 and an effector agent, and the effector agent being covalently or non-covalently conjugated to the transporter peptide.

Embodiment 19. The transporter peptide conjugate of Embodiment 18, wherein the effector agent is at least one selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

Embodiment 20. A composition, comprising
the transporter peptide of any one of Embodiments 1 to 8;
the recombinant transporter peptide conjugate of Embodiment 16; or
the transporter peptide conjugate of Embodiments 18 or 19.

Embodiment 21. The composition of Embodiment 20, further comprising a pharmaceutically acceptable carrier.

Embodiment 22. A method of transporting the composition of Embodiments 20 or 21 to a target, the method comprising binding the transporter peptide, the transporter peptide of the recombinant transporter peptide conjugate, or the transporter peptide of the transporter peptide conjugate to a transferrin receptor.

Embodiment 23. The method of Embodiment 22, wherein the composition has to be transported across a tissue barrier to arrive at the target, and the transferrin receptor is located on a cell of the tissue barrier.

Embodiment 24. The method of Embodiments 22 or 23, wherein binding of the transporter peptide of the composition, the transporter peptide of the transporter peptide conjugate of the composition, or the transporter peptide of the recombinant transporter peptide conjugate of the composition to the transferrin receptor induces transcytosis of the cells, thereby transporting the composition across the tissue barrier to arrive at the target.

Embodiment 25. The method of any one of Embodiments 22 to 24, wherein the tissue barrier is one of a blood-brain barrier (BBB), a mucosal barrier, and a gastrointestinal barrier.

Embodiment 26. The method of any one of Embodiments 22 to 25, wherein the target is a brain cell.

Embodiment 27. The method of any one of Embodiments 22 to 26, wherein the tissue barrier is blood-brain barrier, and the target is a brain cell.

Embodiment 28. The method of Embodiment 22, wherein the target is a cell expressing the transferrin receptor, and the transferrin receptor is located on the target.

Embodiment 29. The method of Embodiments 22 or 28, wherein the target is a cancer cell.

Embodiment 30. The method of Embodiment 29, wherein the cancer is hepatocellular carcinoma, breast cancer, lung cancer, colon cancer, brain cancer, glioma, prostate cancer, ovarian cancer, or leukemia.

Embodiment 31. The method of Embodiments 22 or 29, wherein the target is a tissue cell.

Embodiment 32. The method of Embodiment 31, wherein the tissue is skin, tonsil, tongue, oesophagus, cervix, kidney, placenta, pancreas, testis, anterior pituitary, stomach, breast, or liver.

Embodiment 33. The method of any one of Embodiments 22 to 32, wherein the target is in vivo or in vitro.

Embodiment 34. A method of transporting an effector agent across a tissue barrier of a subject, comprising administering to the subject the recombinant transporter peptide conjugate of Embodiment 16 or the transporter peptide conjugate of Embodiments 18 or 19.

Embodiment 35. The method of Embodiment 34, wherein the recombinant transporter peptide conjugate or the transporter peptide conjugate comprises the effector agent.

Embodiment 36. The method of Embodiment 34 or 35, wherein the tissue barrier is one of a blood-brain barrier, a mucosal barrier, and a gastrointestinal barrier.

Embodiment 37. The method of any one of Embodiments 34 to 36, wherein cells of the tissue barrier express transferrin receptors.

Embodiment 38. The method of any one of Embodiments 34 to 37, wherein the transporter peptide binds to the transferrin receptor, and the binding of the transporter peptide to the transferrin receptor induces transcytosis of the cells, thereby transporting the transporter peptide and the effector agent across the tissue barrier.

Embodiment 39. The method of any one of Embodiments 34 to 38, wherein the recombinant transporter peptide conjugate or the transporter peptide conjugate is administered to the subject by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

Embodiment 40. The method of any one of Embodiments 34 to 39, wherein the effector agent comprised in the recombinant transporter peptide conjugate is selected from the group consisting of peptides, proteins, antibodies, viral particles, liposomes, endosomes, exosomes, ligands, eukaryotic cells, prokaryotic cells, and microspheres.

Embodiment 41. The method of any one of Embodiments 34 to 39, wherein the effector agent comprised in the transporter peptide conjugate is selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

Embodiment 42. The method of any one of Embodiments 34 to 41, wherein the subject is a mammal.

Embodiment 43. The method of any one of Embodiments 34 to 42, wherein the subject is a rodent or a human.

Embodiment 44. A method of treating or preventing a central nervous system (CNS) disease, comprising administering a subject in need thereof a pharmaceutically effective amount of the recombinant transporter peptide conjugate of Embodiment 16 or the transporter peptide conjugate of Embodiments 18 or 19.

Embodiment 45. The method of Embodiment 44, wherein the composition further comprises a pharmaceutically acceptable carrier.

Embodiment 46. The method of Embodiments 44 or 45, wherein the effector agent comprised in the recombinant transporter peptide conjugate or in the transporter peptide conjugate is a therapeutic agent of the CNS disease.

Embodiment 47. The method of any one of Embodiments 44 to 46, wherein the transporter peptide of the recombinant transporter peptide conjugate or the transporter peptide of the transporter peptide conjugate binds to a transferrin receptor on a cell of a tissue barrier of the subject, and the binding of the transporter peptide to the transferrin receptor induces transcytosis of the cell, thereby transporting the transporter peptide and the therapeutic agent of the CNS disease across the tissue barrier.

Embodiment 48. The method of any one of Embodiments 44 to 47, wherein the tissue barrier is one of a blood-brain barrier, a mucosal barrier, and a gastrointestinal barrier.

Embodiment 49. The method of any one of Embodiments 44 to 48, wherein the subject is a mammal.

Embodiment 50. The method of any one of Embodiments 44 to 49, wherein the subject is a rodent or a human.

Embodiment 51. The method of any one of Embodiments 44 to 50, wherein the recombinant transporter peptide conjugate or the transporter peptide conjugate is administered to the subject in need thereof by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

Embodiment 52. The method of any one of Embodiments 44 to 51, wherein the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Embodiment 53. The transporter peptide of any one of Embodiments 1 to 8 for use in delivering an effector agent to a target.

Embodiment 54. Use of the transporter peptide of any one of Embodiments 1 to 8 for the manufacture of a medicament for delivering an effector agent to a target.

Embodiment 55. The use of Embodiments 53 or 54, wherein the transporter peptide is covalently or non-covalently conjugated to the effector agent to form the transporter peptide conjugate of Embodiments 18 or 19.

Embodiment 56. The use of Embodiments 53 or 54, wherein a nucleic acid encoding the transporter peptides and the effector agent is expressed to form the recombinant transporter peptide conjugate of Embodiment 16.

Embodiment 57. The use of any one of Embodiments 53 to 56, wherein the transporter peptide and the effector agent have to cross a tissue barrier to the target, and the transporter peptide binds to a transferrin receptor on a cell of the tissue barrier.

Embodiment 58. The use of Embodiment 57, wherein binding of the transporter peptide to the transferrin receptor induces transcytosis of the cells, thereby transporting the transporter peptide and the effector agent across the tissue barrier to arrive at the target.

Embodiment 59. The use of Embodiments 57 or 58, wherein the tissue barrier is one of a blood-brain barrier, a mucosal barrier, and a gastrointestinal barrier.

Embodiment 60. The use of any one of Embodiments 53 to 56, wherein the target is a cell expressing a transferrin receptor, and the transporter peptide delivers the effector agent to the target by binding to the transferrin receptor expressed on the cell.

Embodiment 61. The use of Embodiment 60, wherein the target is a cancer cell or a tissue cell.

Embodiment 62. The use of Embodiment 61, wherein the cancer is hepatocellular carcinoma, breast cancer, lung cancer, colon cancer, brain cancer, glioma, prostate cancer, ovarian cancer, or leukemia.

Embodiment 63. The use of Embodiment 61, wherein the tissue is skin, tonsil, tongue, oesophagus, cervix, kidney, placenta, pancreas, testis, anterior pituitary, stomach, breast, or liver.

Embodiment 64. The use of any one of Embodiments 53 to 63, wherein the target is in vivo or in vitro.

Embodiment 65. The transporter peptide of any one of Embodiments 1 to 8 for use in transporting an effector agent across a tissue barrier of a subject.

Embodiment 66. Use of the transporter peptide of any one of Embodiments 1 to 8 for the manufacture of a medicament for transporting an effector agent across a tissue barrier of a subject.

Embodiment 67. The use of Embodiments 65 or 66, wherein the transporter peptide is covalently or non-covalently conjugated to the effector agent to form the transporter peptide conjugate of Embodiments 18 or 19.

Embodiment 68. The use of Embodiments 65 or 66, wherein a nucleic acid encoding the transporter peptides and the effector agent is expressed to form the recombinant transporter peptide conjugate of Embodiment 16.

Embodiment 69. The use of any one of Embodiments 65 to 68, wherein the tissue barrier is one of a blood-brain barrier, a mucosal barrier, and a gastrointestinal barrier.

Embodiment 70. The use of any one of Embodiments 65 to 69, wherein one cell of the tissue barrier has a transferrin receptor.

Embodiment 71. The use of any one of Embodiments 65 to 70, wherein the transporter peptide binds to the transferrin receptor, and the binding of the transporter peptide to the transferrin receptor induces transcytosis of the cells, thereby transporting the transporter peptide and the effector agent across the tissue barrier.

Embodiment 72. The transporter peptide of any one of Embodiments 1 to 8 for use in the treatment and/or prevention of a central nervous system (CNS) disease.

Embodiment 73. Use of the transporter peptide of any one of Embodiments 1 to 8 for the manufacture of a medicament for the treatment and/or prevention of a central nervous system (CNS) disease.

Embodiment 74. The use of Embodiments 72 or 73, wherein the transporter peptide is covalently or non-covalently conjugated to the effector agent to form the transporter peptide conjugate of Embodiments 18 or 19.

Embodiment 75. The use of Embodiments 72 or 73, wherein a nucleic acid encoding the transporter peptides and the effector agent is expressed to form the recombinant transporter peptide conjugate of Embodiment 16.

Embodiment 76. The use of Embodiments 74 or 75, wherein the effector agent is a therapeutic agent of the CNS disease.

Embodiment 77. The use of any one of Embodiments 74 to 76, wherein the transporter peptide conjugate or the recombinant transporter peptide conjugate is administered to a subject in need thereof.

Embodiment 78. The use of Embodiment 77, wherein the transporter peptide binds to a transferrin receptor on a cell of a tissue barrier of the subject, and the binding of the transporter peptide to the transferrin receptor induces transcytosis of the cells, thereby transporting the transporter peptide and the therapeutic agent of the CNS disease across the tissue barrier.

Embodiment 79. The use of Embodiment 78, wherein the tissue barrier is one of a blood-brain barrier, a mucosal barrier, and a gastrointestinal barrier.

Embodiment 80. The use of any one of Embodiments 72 to 79, wherein the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Embodiment 81. The use of any one of Embodiments 53 to 55, 57 to 67, 69 to 74, 76 to 80, wherein the effector agent comprised in the transporter peptide conjugate is selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

Embodiment 82. The use of any one of Embodiments 53 to 54, 56 to 66, 68 to 73, 75 to 80, wherein the effector agent comprised in the recombinant transporter peptide conjugate is selected from the group consisting of peptides, proteins, antibodies, viral particles, liposomes, endosomes, exosomes, ligands, eukaryotic cells, prokaryotic cells, and microspheres.

Embodiment 83. The use of any one of Embodiments 65 to 82, wherein the subject is a mammal.

Embodiment 84. The use of any one of Embodiments 65 to 83, wherein the subject is a rodent or a human.

These and other aspects will become apparent from the following description of the preferred embodiment taken in conjunction with the following drawings.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 shows the experimental results of the transporting efficiency of the transporter peptide (PT-034, SEQ ID NO: 19) across Caco-2 cell monolayer.

FIG. 2A shows the in vivo fluorescence imaging of mice after 1, 2, 4, 6, and 24 hours after administration of the transporter peptide (PT-034, SEQ ID NO: 19). FIG. 2B shows the quantification of fluorescence intensity in mouse brain.

FIG. 3A shows the in vivo fluorescence imaging of mice after 0.5, 1, 2, 4, 6, and 24 hours after administration of the transporter peptide (PT-001, SEQ ID NO: 2; PT-025, SEQ ID NO: 11, or PT-031, SEQ ID NO: 17). FIG. 3B shows the quantification of fluorescence intensity in mouse brain.

FIG. 4A shows the in vivo fluorescence imaging of mice after 0.5, 1, 2, 4, 6, and 24 hours after administration of the transporter peptide conjugate (the conjugate of PT-034 and an antibody). FIG. 4B shows the quantification of fluorescence intensity in mouse brain.

FIG. 5A shows the in vivo fluorescence imaging of mice after 0.5, 1, 2, 4, 6, and 24 hours after intravenous or oral administration of the transporter peptide conjugate (the conjugate of PT-034 and an antibody). FIG. 5B shows the fluorescence imaging of mouse brains. FIG. 5C shows the quantification of fluorescence intensity in mouse brain.

FIG. 6A shows the in vivo fluorescence imaging of mice after 0.5, 1, 2, 4, 6, and 24 hours after administration of the transporter peptide conjugate (the conjugate of PT-034 and lipid nanoparticle). FIG. 6B shows the quantification of fluorescence intensity in mouse brain.

### DETAILED DESCRIPTION OF THE PREFERRED EMBODIMENT

The present invention is based, at least in part, on the discovery that the transporter peptides of the present invention can effectively bind to transferrin receptors (TfRs) on a cell. In some embodiments, the transporter peptides of the present invention conjugate covalently or non-covalently to a substance to form a transporter peptide conjugate. In some embodiments, a recombinant transporter peptide conjugate is formed by expressing a nucleic acid encoding the transporter peptides of the present invention and a substance. In the case of the cell being a cell of a tissue barrier, the binding of the transporter peptides to the TfRs induces transcytosis of the cell, thereby delivering the substance conjugated to the transporter peptides across the tissue barrier. In the case of the cell being the target cell, the binding of the transporter peptides to the TfRs directly deliver the substance to the target cell. Therefore, the transporter peptides of the present invention can serve as a drug delivery system. In particular, the transporter peptides of the present invention can be transported across a tissue barrier, especially a blood-brain barrier, by transcytosis and delivery the substance conjugated to the transporter peptides to brain. Therefore, the transporter peptides of the present invention can be used for prevention and/or treatment of CNS diseases.

Specifically, the present invention provides a transporter peptide, consisting of 10 amino acids having the general sequence of

X₁-X₂-X₃-X₄-X₅-X₆-X₇-X₈-X₉-X₁₀

wherein,
X₁ is an acidic amino acid (Asp or Glu) or a polar uncharged amino acid (Asn, Gln, Cys, Ser, Thr, or Tyr);
X₂ is a nonpolar amino acid (Ile, Gly, Ala, Leu, Val, Pro, Phe, Trp, or Met);
X₃ is an arbitrary amino acid (Gly, Ala, Val, Leu, Ile, Phe, Tyr, Trp, His, Asp, Asn, Glu, Gln, Lys, Arg, Ser, Thr, Met, Cys, or Pro);
X₄ is an acidic amino acid (Asp or Glu) or a nonpolar amino acid (Ile, Gly, Ala, Leu, Val, Pro, Phe, Trp, or Met);
X₅ is a nonpolar amino acid (Ile, Gly, Ala, Leu, Val, Pro, Phe, Trp, or Met);
X₆ is an arbitrary amino acid (Gly, Ala, Val, Leu, Ile, Phe, Tyr, Trp, His, Asp, Asn, Glu, Gln, Lys, Arg, Ser, Thr, Met, Cys, or Pro);
X₇ is a basic amino acid (Lys, His, or Arg);
X₈ is an acidic amino acid (Asp or Glu) or a basic amino acid (Lys, His, or Arg);
X₉ is a nonpolar amino acid (Ile, Gly, Ala, Leu, Val, Pro, Phe, Trp, or Met) or a polar uncharged amino acid (Asn, Gln, Cys, Ser, Thr, or Tyr); and
X₁₀ is an acidic amino acid (Asp or Glu) or a polar uncharged amino acid (Asn, Gln, Cys, Ser, Thr, or Tyr).

Preferably and more specifically, the present invention provides a transporter peptide, consisting of 10 amino acids having the amino acid sequence of SEQ ID NO: 1:

(SEQ ID NO: 1)
X₁-Ile-X₃-Val-Leu-X₆-Lys-X₈-X₉-X₁₀,

wherein
X₁ is Asp, Glu, Asn, Gln, Cys, Ser, Thr, or Tyr;
the Ile at the second amino acid site of SEQ ID NO: 1 can be replaced by Gly, Ala, Leu, Val, Pro, Phe, Trp, or Met;
X₃ is Gly, Ala, Val, Leu, Ile, Phe, Tyr, Trp, His, Asp, Asn, Glu, Gln, Lys, Arg, Ser, Thr, Met, Cys, or Pro;
the Val at the fourth amino acid site of SEQ ID NO: 1 can be replaced by Asp, Glu, Ile, Gly, Ala, Leu, Pro, Phe, Trp, or Met;
the Leu at the fifth amino acid site of SEQ ID NO: 1 can be replaced by Ile, Gly, Ala, Val, Pro, Phe, Trp, or Met;
X₆ is Gly, Ala, Val, Leu, Ile, Phe, Tyr, Trp, His, Asp, Asn, Glu, Gln, Lys, Arg, Ser, Thr, Met, Cys, or Pro;
the Lys at the seventh amino acid site of SEQ ID NO: 1 can be replaced by His or Arg;
X₈ is Asp, Glu, Lys, His, or Arg;
X₉ is Ile, Gly, Ala, Leu, Val, Pro, Phe, Trp, Met, Asn, Gln, Cys, Ser, Thr, or Tyr; and
X₁₀ is Asp, Glu, Asn, Gln, Cys, Ser, Thr, or Tyr.

Preferably and in some embodiments, the amino acid sequence of SEQ ID NO: 1 comprises, but is not limited to, the sequence any one of SEQ ID NO: 2 to SEQ ID NO: 30 and variant sequences at least 70% homologous to one of SEQ ID NO: 2 to SEQ ID NO: 30 and being able to bind to the transferrin receptor. Preferably and in some embodiments, Preferably and in some embodiments, the variant sequences are at least 70% homologous to one of SEQ ID NO: 2 to SEQ ID NO: 30. Preferably and in some embodiments, the variant sequences are at least 70%, least 75%, least 80%, least 85%, least 900, least 950 homologous to one of SEQ ID NO: 2 to SEQ TD NO: 30. Each possibility represents a separate embodiment of the invention. SEQ ID NO: 2 to SEQ TD NO: 30 are set out in Table 1.

To determine the percent identity of two sequences, the sequences are aligned for optimal comparison purposes (e.g., gaps can be introduced in the sequence of a first amino acid sequence for optimal alignment with a second amino acid nucleotide sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

TABLE 1
Peptide names and sequences of SEQ ID NO: 2
to SEQ ID NO: 30.

| SEQ ID NO | Peptide Name | Sequence |
|---|---|---|
| 2 | PT-001 | DISVLMKEYD |
| 3 | PT-004 | DIDVLMKEYD |
| 4 | PT-008 | DIHVLMKEYD |
| 5 | PT-011 | DILVLMKEYD |
| 6 | PT-014 | DIPVLMKEYD |
| 7 | PT-017 | DITVLMKEYD |
| 8 | PT-022 | DISLLMKEYD |
| 9 | PT-023 | DISVIMKEYD |
| 10 | PT-024 | DISVVMKEYD |
| 11 | PT-025 | DISVLDKEYD |
| 12 | PT-026 | DISVLNKEYD |
| 13 | PT-027 | DISVLCKEYD |
| 14 | PT-028 | DISVLKKEYD |
| 15 | PT-029 | DISVLEKEYD |
| 16 | PT-030 | DISVLQKEYD |
| 17 | PT-031 | DISVLMKEYN |
| 18 | PT-033 | DISVLMKEYE |
| 19 | PT-034 | DISVLMKEYQ |
| 20 | PT-051 | DISVLSKEYD |
| 21 | PT-054 | DISVLMKDYD |
| 22 | PT-055 | DISVLMKEFD |
| 23 | PT-056 | DISVLMKESD |
| 24 | PT-057 | DISVLMKETD |
| 25 | PT-062 | NISVLMKEYD |
| 26 | PT-063 | DISELMKEYD |
| 27 | PT-064 | DISVLLKEYD |
| 28 | PT-065 | DISVLMKHYD |
| 29 | PT-066 | DITVLCKEYN |
| 30 | PT-067 | DIHVLDKEYD |

Preferably and in some embodiments, the amino acid of SEQ ID NO: 1 has the binding affinity to a TfR.

As used herein, the term “peptide” refers to a molecular chain of amino acids, including both L-forms and D-forms. The amino acids, if required, can be modified in vivo or in vitro, for example by manosylation, glycosylation, amidation (specifically C-terminal amides), carboxylation or phosphorylation with the stipulation that these modifications must preserve the biological activity of the original molecule. In addition, peptides can be part of a chimeric protein. As used in this article, the term “transporter peptide” is described from the application aspect of the peptide and generally refers to a peptide that can bind to a specific receptor to induce a transport effect. A transporter peptide is also a targeting peptide.

Functional derivatives of the peptides are also included in the present invention. Functional derivatives are meant to include peptides which differ in one or more amino acids in the overall sequence, which have deletions, substitutions, inversions or additions. Amino acid substitutions which can be expected not to essentially alter biological and immunological activities have been described. Amino acid replacements between related amino acids or replacements which have occurred frequently in evolution include, inter alia Ser/Ala, Ser/Gly, Asp/Gly, Asp/Asn and Ile/Val.

The peptides according to the invention can be produced synthetically or by recombinant DNA technology. Methods for producing synthetic peptides are well known in the art.

As used herein, the nomenclature used to describe peptides of the present invention follows the conventional practice in which the amino group (N-terminus) and/or the 5′ are presented to the left and the carboxyl group (C-terminus) and/or 3′ are presented to the right.

As used herein, the term “the amino acid site” refers to a specific amino acid position in the peptide, calculated from the N-terminus of the peptide. For example, the first amino acid site (X₁) refers to the position of the first amino acid based on the N-terminal number of the peptide.

As used herein, the term “an arbitrary amino acid” refers to any amino acid selected from the 20 basic amino acids. As used herein, the term “basic amino acids” refers to the following 20 amino acids: Glycine (Gly, G), Alanine (Ala, A), Valine (Val, V), Leucine (Leu, L), Isoleucine (Ile, I), Phenylalanine (Phe, F), Tyrosine (Tyr, Y), Tryptophan (Trp, W), Histidine (His, H), Aspartic acid (Asp, D), Asparagine (Asn, N), Glutamic acid (Glu, E), Glutamine (Gln, Q), Lysine (Lys, K), Arginine (Arg, R), Serine (Ser, S), Threonine (Thr, T), Methionine (Met, M), Cysteine (Cys, C), and Proline (Pro, P). The symbols used to represent amino acids herein are the same as the amino acid abbreviations used by those skilled in the art. Unless otherwise defined, as used herein, the term “acidic amino acid” refers to Asp or Glu, the term “basic amino acid” refers to His, Arg, or Lys, the term “polar uncharged amino acid” refers to Asn, Gln, Cys, Ser, Thr or Tyr, and the term “non-polar amino acid” refers to Gly, Ala, Ile, Leu, Val, Pro, Phe, Trp or Met. When an amino acid is not specifically indicated to be a right- or left-handed, the amino acid may be a left-handed amino acid or a right-handed amino acid, unless the context clearly indicates that the amino acid is a specific isomer.

As used herein, the term “transferrin receptor (TfR)” refers to a type II transmembrane glycoprotein having a molecular weight of 90 kDa and found forming a homodimer (180 kDa) linked by disulfide bond on the surface of the cell on which it located. The amino acid sequence of human TfR is as shown as the protein number P02786 on the Uniprot website (https://www.uniprot.org/uniprotkb/P02786/entry). Each extracellular domain of TfR is consisted of three domains including apical domain (189th to 383rd amino acid residues), protease like domain (122nd to 188th amino acid residues and 384th to 606th amino acid residues), and helical domain (607th to 760th amino acid residues). The primary function of TfR is to bind to transferrin (Tf), a protein that carries iron in the bloodstream, and facilitate the uptake of iron into cells. The transferrin binding region is major on the TfR1 dimer surface of the helical domain and a small part of protease-like domain. The binding of TfR to transferrin induces transcytosis of the cell on which the TfR is located, thereby transporting the Tf and the iron carried by the Tf into the cell. Transferrin receptors are commonly found on various tissue barriers, including blood-brain barrier, gastrointestinal barrier, and mucosal barrier. In addition, transferrin receptors have been found to have relatively high expression levels in rapidly proliferating cells (e.g., cancer cells), such as those associated with hepatocellular carcinoma, breast cancer, lung cancer, colon cancer, brain cancer, glioma, prostate cancer, ovarian cancer, or leukemia. Transferrin receptors have also been found to be expressed in cells of some tissues, including, but not limited to, skin, tonsil, tongue, oesophagus, cervix, kidney, placenta, pancreas, testis, anterior pituitary, stomach, breast, or liver.

The present invention also provides nucleic acids encoding the transporter peptides disclosed herein. Preferably and in some embodiments, the nucleic acid is deoxyribonucleic acid (DNA). Preferably and in some embodiments, the nucleic acid is ribonucleic acid (RNA).

As used herein, the term “nucleotide” refers to a monomer comprising a nitrogenous base connected to a sugar phosphate that comprises a sugar, such as ribose or 2′-deoxyribose, connected to one or more phosphate groups. “Polynucleotide” and “nucleic acid” refer to a polymer comprising more than one nucleotide monomer, in which said monomers are often connected by sugar-phosphate linkages of a sugar-phosphate backbone. A polynucleotide need not comprise only one type of nucleotide monomer. For example, the nucleotides comprising a given polynucleotide may be only ribonucleotides, only 2′-deoxyribonucleotides, or a combination of both ribonucleotides and 2′-deoxyribonucleotides. Polynucleotides include naturally occurring nucleic acids, such as deoxyribonucleic acid (“DNA”) and ribonucleic acid (“RNA”), as well as nucleic acid analogs comprising one or more non-naturally occurring monomer. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form, but in which “T” replaces “U.” The term “recombinant nucleic acid” refers to a polynucleotide or nucleic acid having sequences that are not naturally joined together. A recombinant nucleic acid may be present in the form of a vector.

The polynucleotide sequences encoding any one of the transporter peptides disclosed herein are derived from the amino acid sequences of the transporter peptides by replacing each amino acid with a three-nucleotide codon, including every degenerate codon, listed in the genetic code table. For example, each proline of the amino acid sequences of the peptides can be independently encoded by the codons CCA, CCC, CCG, or CCT.

The present invention also provides a vector comprising the nucleic acids encoding the transporter peptides disclosed herein. Preferably and in some embodiments, the vector is an adeno-associated virus (AAV) vector. Preferably and in some embodiments, the vector further comprises a nucleic acid encoding an effector agent. More preferably and in some preferred embodiments, the effector agent is at least one selected from the group consisting of peptides, proteins, antibodies, viral particles, liposomes, endosomes, exosomes, ligands, eukaryotic cells, prokaryotic cells, and microspheres.

As used herein, the term “vector” refers to a DNA molecule used as a vehicle to carry foreign DNA into a host organism, typically a bacterium, yeast, or mammalian cell. Examples of vectors include, but is not limited to, plasmids, bacteriophages, bacterial artificial chromosomes (BACs), yeast artificial chromosomes (YACs), cosmids, shuttle vectors, expression vecotors, retroviral vectors, and adenoviral vectors.

As used herein, the term “adeno-associated virus (AAV)” refers to a small, non-enveloped virus belonging to the family Parvoviridae and the genus Dependoparvovirus. AAV is non-replicating in infected cells and therefore not associated with any known disease in humans and is considered relatively safe for use as a gene delivery vehicle. As used herein, the term “adeno-associated virus (AAV) vector” refers to vectors derived from the wild-type adeno-associated virus by removing most of its viral genes and replacing them with the gene of interest. AAV vectors are used to introduce specific genes or genetic material into target cells or tissues and commonly used in gene therapy and molecular biology research.

As used herein, “an effector agent” refers to any molecule that imparts an effect on a target inside the tissue barrier, and as disclosed herein the target may be target cells or extracellular molecules.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2, single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

The present invention also provides a recombinant transporter peptide conjugate expressed by the vector described herein.

The present invention also provides a recombinant host cell comprising one component selected from the transporter peptide described herein, the nucleic acid described herein, and the vector described herein.

The present invention also provides a transporter peptide conjugate, consisting of any one of the transporter peptides described herein and an effector agent being covalently or non-covalently conjugated to the transporter peptide. Preferably and in some embodiments, the effector agent is at least one selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

The present invention also provides a composition, comprising at least one of any one of the transporter peptide described herein, the recombinant transporter peptide conjugate described herein, and the transporter peptide conjugate described herein. Preferably and in some embodiments, the composition further comprises a pharmaceutically acceptable carrier.

As used herein, “pharmaceutically acceptable carrier” or “pharmaceutically acceptable excipient” includes any and all solvents, dispersion media, coatings, antibacterial and antifungal agents, isotonic and absorption enhancing or delaying agents, and other excipients or additives that are physiologically compatible. In specific embodiments, the carrier is suitable for intranasal, intravenous, intramuscular, intradermal, subcutaneous, parenteral, oral, transmucosal or transdermal administration. Depending on the route of administration, the active compound may be coated in a material to protect the compound from the action of acids and other natural conditions which may inactivate the compound. The use of such media and agents for pharmaceutically active substances is well known in the art.

The present invention also provides a method of transporting the composition described herein to a target, the method comprising binding the transporter peptide, the transporter peptide of the recombinant transporter peptide conjugate, or the transporter peptide of the transporter peptide conjugate to a transferrin receptor. The present invention also provides any one of the transporter peptide described herein for use in delivering an effector agent to a target. The present invention also provides use of any one of the transporter peptide described herein for the manufacture of a medicament for delivering an effector agent to a target. The target is in vivo or in vitro.

Preferably and in some embodiments, the composition has to be transported across a tissue barrier to arrive at the target, and the transferrin receptor is located on a cell of the tissue barrier. Preferably and in some embodiments, binding of the transporter peptide of the composition, the transporter peptide of the transporter peptide conjugate of the composition, or the transporter peptide of the recombinant transporter peptide conjugate of the composition to the transferrin receptor induces transcytosis of the cells, thereby transporting the composition across the tissue barrier to arrive at the target. Preferably and in some embodiments, the tissue barrier is one of a blood-brain barrier (BBB), a mucosal barrier, and a gastrointestinal barrier. Preferably and in some embodiments, the target is a brain cell. More preferably and in some preferred embodiments, the tissue barrier is blood-brain barrier, and the target is a brain cell.

Preferably and in some embodiments, the target is a cell expressing the transferrin receptor, and the transferrin receptor is located on the target. Preferably and in some embodiments, the target is a cancer cell. More preferably and in some preferred embodiments, the cancer is hepatocellular carcinoma, breast cancer, lung cancer, colon cancer, brain cancer, glioma, prostate cancer, ovarian cancer, or leukemia. Preferably and in some embodiments, the target is a tissue cell. More preferably and in some preferred embodiments, the tissue is skin, tonsil, tongue, oesophagus, cervix, kidney, placenta, pancreas, testis, anterior pituitary, stomach, breast, or liver.

The present invention also provides a method of transporting an effector agent across a tissue barrier of a subject, comprising administering to the subject the recombinant transporter peptide conjugate described herein or the transporter peptide conjugate described herein. The present invention also provides any one of the transporter peptides described herein for use in transporting an effector agent across a tissue barrier of a subject. The present invention also provides use of any one of the transporter peptides described herein for the manufacture of a medicament for transporting an effector agent across a tissue barrier of a subject.

Preferably and in some embodiments, the recombinant transporter peptide conjugate or the transporter peptide conjugate comprises the effector agent. Preferably and in some embodiments, the tissue barrier is one of a blood-brain barrier, a mucosal barrier, and a gastrointestinal barrier. Preferably and in some embodiments, cells of the tissue barrier express transferrin receptors. Preferably and in some embodiments, the transporter peptide binds to the transferrin receptor, and the binding of the transporter peptide to the transferrin receptor induces transcytosis of the cells, thereby transporting the transporter peptide and the effector agent across the tissue barrier. Preferably and in some embodiments, the recombinant transporter peptide conjugate or the transporter peptide conjugate is administered to the subject by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery. Preferably and in some embodiments, the effector agent comprised in the recombinant transporter peptide conjugate is selected from the group consisting of peptides, proteins, antibodies, viral particles, liposomes, endosomes, exosomes, ligands, eukaryotic cells, prokaryotic cells, and microspheres. Preferably and in some embodiments, the effector agent comprised in the transporter peptide conjugate is selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules. Preferably and in some embodiments, the subject is a mammal. More preferably and in some preferred embodiments, the subject is a rodent or a human.

The present invention also provides a method of treating or preventing a central nervous system (CNS) disease, comprising administering a subject in need thereof a pharmaceutically effective amount of the recombinant transporter peptide conjugate described herein or the transporter peptide conjugate described herein. The present invention also provides any one of the transporter peptide described herein for use in the treatment and/or prevention of a central nervous system (CNS) disease. The present invention also provides use of any one of the transporter peptide described herein for the manufacture of a medicament for the treatment and/or prevention of a central nervous system (CNS) disease.

Preferably and in some embodiments, the composition further comprises a pharmaceutically acceptable carrier. Preferably and in some embodiments, the effector agent comprised in the recombinant transporter peptide conjugate or in the transporter peptide conjugate is a therapeutic agent of the CNS disease. Preferably and in some embodiments, the transporter peptide of the recombinant transporter peptide conjugate or the transporter peptide of the transporter peptide conjugate binds to a transferrin receptor on a cell of a tissue barrier of the subject, and the binding of the transporter peptide to the transferrin receptor induces transcytosis of the cell, thereby transporting the transporter peptide and the therapeutic agent of the CNS disease across the tissue barrier. Preferably and in some embodiments, he tissue barrier is one of a blood-brain barrier, a mucosal barrier, and a gastrointestinal barrier. Preferably and in some embodiments, the subject is a mammal. More preferably and in some preferred embodiments, the subject is a rodent or a human. Preferably and in some embodiments, the recombinant transporter peptide conjugate or the transporter peptide conjugate is administered to the subject in need thereof by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery. Preferably and in some embodiments, the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

Formulations suitable for administration of the present invention may comprise, possibly among other things well known to those of skill in the art: aqueous and non-aqueous solutions, antioxidants, bacteriostats, buffers, solutes that affect isotonicity, preservatives, solubilizers, stabilizers, suspending agents, thickening agents, or a combination thereof.

In addition or in the alternative, formulations suitable for administration of the present invention may comprise, possibly among other things well known to those of skill in the art: gels, PEG such as PEG 400, propylene glycol, saline, sachets, water, other appropriate liquids known in the art, or a combination thereof.

Also in the addition or in the alternative, formulations suitable for administration of the present invention may comprise, possibly among other things well known to those of skill in the art: binders, buffering agents, calcium phosphates, cellulose, colloids, such as colloidal silicon dioxide, colorants, diluents, disintegrating agents, dyes, fillers, flavoring agents, gelatin, lactose, magnesium stearate, mannitol, microcrystalline gelatin, moistening agents, paraffin hydrocarbons, pastilles, polyethylene glycols, preservatives, sorbitol, starch, such as corn starch, potato starch, or a combination thereof, stearic acid, sucrose, talc, triglycerides, or a combination thereof.

Also in addition or in the alternative, formulations suitable for administration of the present invention may comprise, possibly among other things well known to those of skill in the art: alcohol such as benzyl alcohol or ethanol, benzalkonium chloride, buffers such as phosphate buffers, acetate buffers, citrate buffers, or a combination thereof, carboxymethylcellulose or microcrystalline cellulose, cholesterol, dextrose, juice such as grapefruit juice, milk, phospholipids such as lecithin, oil such as vegetable, fish, or mineral oil, or a combination thereof, other pharmaceutically compatible carriers known in the art, or a combination thereof.

Also in the addition or in the alternative, formulations suitable for administration of the present invention may comprise, possibly among other things well known to those of skill in the art: biodegradables such as poly-lactic-coglycolic acid (PLGA) polymer, other entities whose degradation products can quickly be cleared from a biological system, or a combination thereof.

Formulations of the present invention may be administered in unit-dose form, multi-dose form, or a combination thereof. They may be packaged in unit-dose containers, multi-dose containers, or a combination thereof. The present invention may exist in ampoules, cachets, capsules, granules, lozenges, powders, tablets, vials, emulsions, including but not limited to acacia emulsions, suspensions, or a combination thereof.

As used herein, an “effective amount” or a “sufficient amount” of a substance is that amount sufficient to effect beneficial or desired results, including clinical results, and, as such, an “effective amount” depends upon the context in which it is being applied. In the context of administering an immunogenic composition, the effective amount is an immunogenically effective amount, which contains sufficient immunogenic composition of the present invention to elicit an immune response. In the context of administering a pharmaceutical composition, the effective amount is a pharmaceutically effective amount, which contains sufficient pharmaceutical composition of the present invention to maintain or produce a desired physiological result. An effective amount can be administered in one or more doses.

As used herein, the term “pharmaceutically effective amount” refers to an amount capable of or sufficient to maintain or produce a desired physiological result, including but not limited to treating, reducing, attenuating, eliminating, suppressing, substantially preventing, or prophylaxing, or a combination thereof, a disease, disorder, or combination thereof. A pharmaceutically effective amount may comprise one or more doses administered sequentially or simultaneously. Those skilled in the art will know to adjust doses of the present invention to account for various types of formulations, including but not limited to slow-release formulation. As used herein, the term “prophylactic” refers to a composition capable of substantially preventing or prophylaxing any aspect of a disease, disorder, or combination thereof. As used herein, the term “therapeutic” refers to a composition capable of treating, reducing, halting the progression of, slowing the progression of, beneficially altering, eliminating, or a combination thereof, any aspect of a disease, disorder, or combination thereof.

The term “dose” as used herein in reference to a composition refers to a measured portion of the composition taken by (administered to or received by) a subject at any one time.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, pig. In some embodiments, the subject is a human. Human subjects may also include fetuses.

As used herein, the terms “subject,” refers to any subject, particularly a mammalian subject, for whom therapy is desired, for example, a human.

The term “treat,” “treating,” or “treatment” as used herein encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevent,” “preventing,” or “prevention” refers to being able to substantially preclude, avert, obviate, forestall, stop, hinder, or a combination thereof, any aspect of a disease, condition, or combination thereof from happening, especially by advance action.

In some embodiments, the oligonucleotide therapeutics and/or the composition of the present invention may be administered to subjects by a variety of administration modes, including by intradermal, intramuscular, subcutaneous, intravenous, intra-atrial, intra-articular, intraperitoneal, parenteral, oral, rectal, intranasal, intrapulmonary, and transdermal delivery, or topically to the eyes, ears, skin or mucous membranes. Alternatively, the antigen may be administered ex-vivo by direct exposure to cells, tissues or organs originating from a subject (autologous) or another subject (allogeneic), optionally in a biologically suitable, liquid or solid carrier.

The meaning of the technical and scientific terms as described herein can be clearly understood by a person of ordinary skill in the art.

As used herein, the term “about,” “around,” or “approximately” when combined with a value refers to plus and minus 10% of the reference value. For example, a length of about 1000 nanometers (nm) refers to a length of the range of 900 nm to 1100 nm.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

The animal experiment protocols of each Example disclosed herein have been reviewed and approved (Document Number: IACUC #2023-R403-006) by the Animal Experiment Ethics Committee of the Development Center for Biotechnology (DCB) (Taipei, Taiwan).

### Example 1 Binding Ability Assay of the Transporter Peptides to TfR

In this Example, the binding affinity of the transporter peptides disclosed herein to TfR was analyzed by fiber optic particle plasmon resonance (FOPPR). In this Example, different amino acid sites of the transporter peptide were further substituted with different amino acids of the same property to anaylze binding affinity of these variant peptides to the transferrin receptor.

### Materials and Methods

To determine the affinity of the targeting peptide to TfR1, a fiber optic particle plasmon resonance (FOPPR) affinity study was performed according to the manufacturer's instruction. Briefly, 80 μL of tested peptide (10 μg/ml) dissolved in PBS buffer (pH 7.4) was pipetted into the EDC/NHS preactivated surface of standard chip NanoAu-MM (Taipei, Taiwan) for peptide conjugating to the carboxyl group of the surface sensor chip. TfR protein (Acro Biosystems. Cat. No. CD1-H5243, Newark, DE, USA) was loading to the chip with at the concentrations of 0.5 ng/mL, 1 ng/mL, 10 ng/mL, 100 ng/mL, 500 ng/mL, and 1000 ng/mL, respectively. Based on the light intensity change, the molecules interaction parameters of K_{D} were calculated by the FOPPR system D200 (Id M3) (Taipei, Taiwan).

### Results

The affinity of the transporter peptides disclosed herein to the transferrin receptor is expressed by K_{D} (Kinetic data) value. The higher the K_{D} value, the weaker the binding between the two molecules. On the other hand, the lower the K_{D} value, the stronger the binding between the two molecules (K_{D} value <300 nM means the tested peptides have strong binding affinity to TfR). As shown in Table 2, each transporter peptide (SEQ ID NO: 2 to SEQ ID NO: 30) disclosed herein have strong binding affinity to TfR. The results demonstrated that each site of the transporter peptides disclosed herein (SEQ ID NO: 1) can be substituted by several specific amino acids without loss of binding affinity to TfR.

TABLE 2
Binding affinities of the transporter peptides to TfR

| SEQ ID NO | Peptide name | K_{D} (nM) |
|---|---|---|
| 2 | PT-001 | 70.98 |
| 3 | PT-004 | 18.50 |
| 4 | PT-008 | 8.07 |
| 5 | PT-011 | 3.23 |
| 6 | PT-014 | 52.28 |
| 7 | PT-017 | 4.77 |
| 8 | PT-022 | 36.56 |
| 9 | PT-023 | 22.80 |
| 10 | PT-024 | 8.22 |
| 11 | PT-025 | 0.43 |
| 12 | PT-026 | 26.04 |
| 13 | PT-027 | 0.27 |
| 14 | PT-028 | 5.14 |
| 15 | PT-029 | 3.62 |
| 16 | PT-030 | 10.27 |
| 17 | PT-031 | 4.45 |
| 18 | PT-033 | 261.37 |
| 19 | PT-034 | 39.34 |
| 20 | PT-051 | 69.99 |
| 21 | PT-054 | 59.32 |
| 22 | PT-055 | 6.08 |
| 23 | PT-056 | 4.1 |
| 24 | PT-057 | 1.92 |
| 25 | PT-062 | 1.30 |
| 26 | PT-063 | 16.55 |
| 27 | PT-064 | 10.47 |
| 28 | PT-065 | 0.57 |
| 29 | PT-066 | 86.09 |
| 30 | PT-067 | 0.45 |

### Example 2. In Vitro Transcytosis Analysis of the Transporter Peptide

In this example, the transport efficiency of the transporter peptide disclosed herein to the Caco-2 cell monolayer was tested to analyze the penetration effect of the transporter peptide on the tissue barrier, and to analyze the ability of the transporter peptide to induce transcytosis of the cell.

### Materials and Methods

### Cell Culture

Caco-2 cells were plated at 2×10⁴ cells/cm² on 24-transwell inserts (BD Falcon, 353495) in DMEM medium with 10% FBS and cultured for 21 days to form a monolayer. Monolayers were then confirmed by transepithelial electrical resistance (TEER), and the monolayers which have at least 500-600 Ω cm² of TEER were qualified monolayers for the following transcytosis experiment.

### High Performance Liquid Chromatography (HPLC)

HPLC was conducted by using a Water ACQUITY Arc system. The chromatographic separation was performed on a C18 column (Waters XSelect HSS T3 5 m, 4.6 mm×250 mm). Column temperature was maintained and operated at 40° C. The mobile phase consisted of A-0.1% trifluoroacetic acid in water and B-100% methanol. Separation was performed according to the following gradient program: 0 min-10% B, 6 min-30% B, 12 min-50% B, 18 min-90% B, 23 min-10% B. The flow rate of the mobile phase was at 1 mL/min, the injection volume of the sample was 20 μL, and the total run time was within 30 mins.

### In Vitro Transcytosis Analysis

The caco-2 monolayers with TEER at 680 Ω cm² were equilibrated in DMEM medium (serum free). Ten (10) M of PT-034 (SEQ ID NO: 19) was applied to the top of Caco-2 monolayers at 37° C. Samples of both upper and lower solution were collected at 0, 0.25, 0.5, 1, 2, 6 hours. Peptides were purified from the sample solution by solid phase extraction (SPE) using C-18 tips (Cat. no. 87784, Pierce, Waltham, Massachusetts, USA). Each sample was supplemented with a final concentration of 11.43 μM PT-034 as internal control. 300 μL sample was absorbed by the C18 tip and eluted by 100 μL methanol. The sample was then subjected to HPLC analysis. The HPLC peak area was calculated at retention time of 16.9 minutes, and a standard curve was plotted for calculating the contents of the transporter peptide in the sample solutions. The concentrations of the transporter peptide in the upper and lower soultions were calculated to determine the proportion of the transporter peptide penetrating the Caco-2 cell monolayer.

### Results

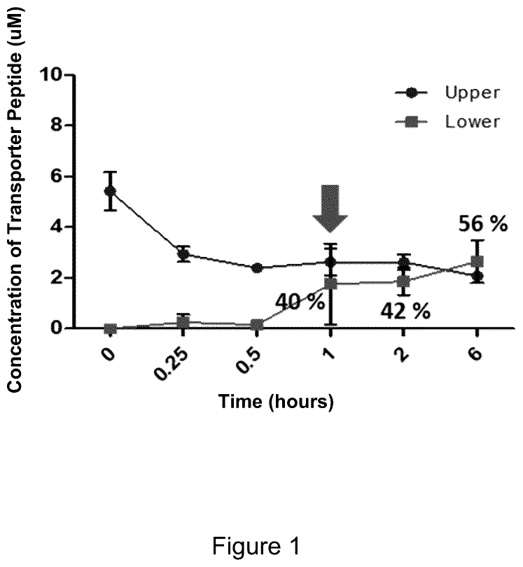

As shown in FIG. 1, about 8%, 40%, 42%, and 56% of the transporter peptide (SEQ ID NO: 19) penetrated the Caco-2 cell monolayer at 0.25, 1, 2, and 6 hours, respectively. The results indicate that the transporter peptide of the present invention has the ability to transport across tissue barriers and effectively induce transcytosis of the cells.

### Example 3. In Vivo Imaging System (IVIS) Analysis of Transporter Peptides

In this Example, the transporter peptide disclosed herein was administered to mice, and the distribution of the transporter peptide in the mice was detected with an in vivo fluorescence imaging system (IVIS).

### Materials and Methods

### Conjugation of Peptides to Fluorescent Dye

One (1) mg of each transporter peptides PT-001 (SEQ ID NO: 2), PT-025 (SEQ ID NO: 11), PT-034 (SEQ ID NO: 19), and PT-031 (SEQ ID NO: 17) was dissolved in 1 mL of buffer containing 50 mM sodium bicarbonate/bicarbonate to a final concentration of 1 mg/mL. Fifty (50) L of 10 mg/mL fluorescent dye (VivoTag 680 XL, PerkinElmer, USA) was added to the 1 mL transporter peptide solution, and the mixture was incubated at room temperature in the dark for 1 hour. The dye-labeled product was filtered with a 3 KDa centrifugal filter device (Amicon Ultra-0.5 Centrifugal filter devices, Millipore, USA) and centrifuged at 14,000×g for 15 minutes. The fluorescent dye-conjugated transporter peptide was diluted with 1×PBS to a final volume of 0.2 mL.

### Animal Trail

BALB/c mice (BioLASCO Taiwan Co., Ltd, Taipei, Taiwan) at the age of 7-8 weeks were divided into a control group (n=1) and a test group (n=3). The mouse in the control group was administered 50 μL of fluorescent dye via tail vein injection. Mice in the group were administered 50 μL of the transporter peptide conjugated with fluorescent dye obtained above via tail vein injection. Mice were photographed using an optical imaging system (U-OI, MlLabs, Netherlands) at 0.5, 1, 2, 4, 6 and 24 hours after the injection for IVIS imaging analysis, with the excitation wavelength of 631 nm, the emission wavelength of 710 nm, and 10-second exposure time. Fluorescence signals of mouse brains were further quantified.

### Results

### 3.1 In Vivo Distribution of PT-034

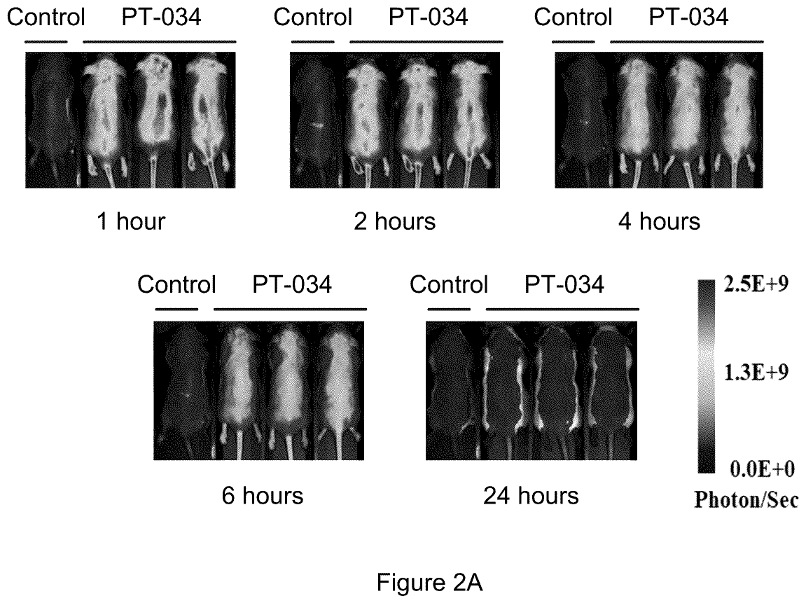

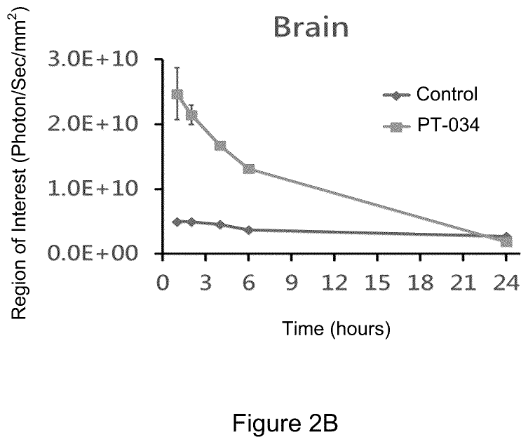

As shown in FIG. 2A, at 1, 2, 4, and 6 hours after injection, the fluorescent signal was enriched in the brain and spine of the mice in the test group, in which 10 mg/kg of the fluorescent dye-conjugated transporter peptide PT-034 (SEQ ID NO: 19) was administered to the mice. In contrast, the mouse in the control group (Dye) did not have such a fluorescence distribution. Then, fluorescence signals of mouse brains were further quantified. As shown in FIG. 2B, compared with the control group, a high level of fluorescent signal was observed in the brains of mice in the test group after 1 hour of the injection, with around 5 hours of half-life of the fluorescence in the mouse brains. The results prove that the transporter peptide disclosed herein has the effect of targeting the brain and spine and can be used as an effective drug delivery system.

### 3.2 In Vivo Distribution of PT-001, PT-025, and PT-031

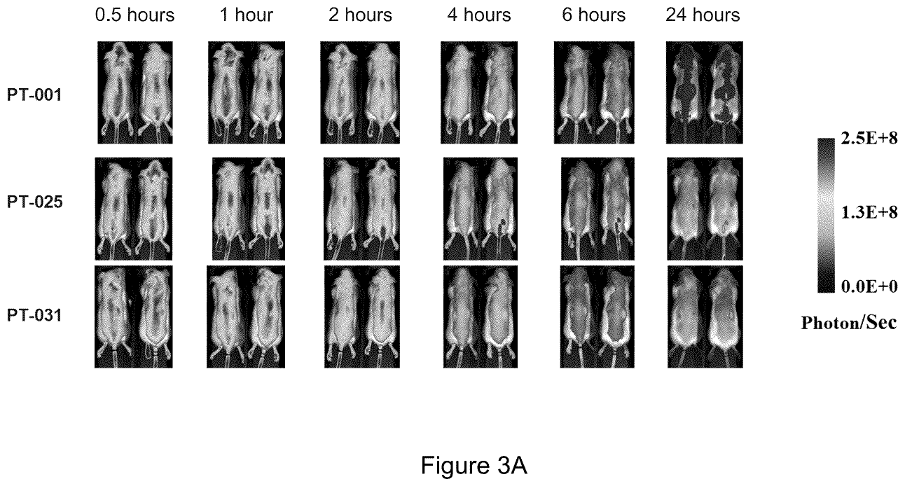

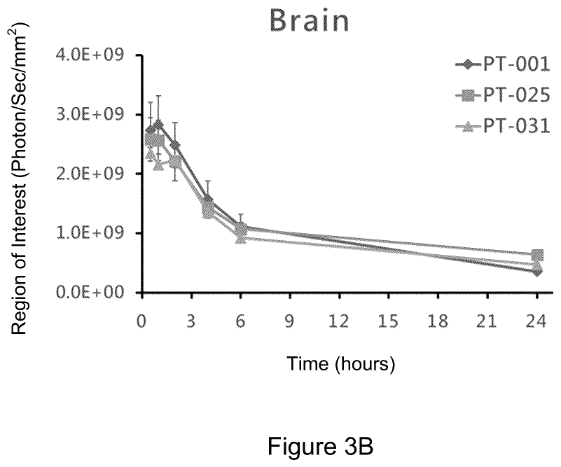

As shown in FIG. 3A, at 0.5, 1, 2, 4, and 6 hours after injection, the fluorescent signals were enriched in the brain and spine of the mice in the test groups, in which 1 mg/kg of one of the fluorescent dye-conjugated transporter peptides PT-001 (SEQ ID NO: 2), PT-025 (SEQ ID NO: 11), and PT-031 (SEQ ID NO: 17) was administered to the mice. Then, fluorescence signals of mouse brains were further quantified. As shown in FIG. 3B, high levels of fluorescent signals were observed in the brains of mice in the test groups after 0.5 hour of the injection, with around 5 hours of half-life of the fluorescence in the mouse brains. The results prove that the transporter peptides (SEQ ID NOs: 2 to 30) having different amino acid substitutions on different amino acid sites of the transporter peptide (SEQ ID NO: 1) of the present invention all have the effect of targeting the brain and spine, and can therefore be used as an effective drug delivery system.

### Example 4. Analysis of Transporting Efficiency to the Brain of a Transporter Peptide

In this Example, the transporter peptide disclosed herein was proved to have the effect of transporting across the blood-brain barrier by analysis the transport efficiency of the transporter peptide in the brain.

### Materials and Methods

Mice (n=2) were administered with 10 mg/kg of the transporter peptide (PT-034, SEQ ID NO: 19) via tail vein injection. Brain tissue was collected one hour after the injection and triturated in 1.3 mL of 50 mM sodium bicarbonate/bicarbonate buffer. The brain tissue grinding fluid was centrifuged at 3000 rpm for 5 minutes, the supernatant was taken and further centrifuged at 14000 rpm for 15 minutes. Zero-point-four (0.4) mL of ice-cold methanol was added to 0.1 mL of the brain tissue grinding supernatant. The mixture was shook for 10 minutes, and then centrifuge at 14,000 rpm for 15 minutes. The brain tissue supernatant was then subjected to HPLC analysis whose method is as described in Example 2. The HPLC peak area was calculated at the retention time of 16.9 minutes, and a standard curve was plotted to calculate the content of the transporter peptide in each sample.

### Results

As shown in Table 3, in each of the intact brain tissues (brain tissue-1 and brain tissue-2) of the mice in the test group, the contents of transit peptide (PT-034) were 25.5 g and 25.9 μg, respectively. The content was converted into the percentage of injection dose per gram of brain tissue (% ID/g of brain, injection dose/brain tissue weight), which are 22.09% and 23.48% respectively, indicating that the average transport efficiency of the transporter peptide to the brain is approximately 22.79% ID/g. The results show that the transporter peptide disclosed herein has the effect of transporting across the blood-brain barrier and reaching the brain.

TABLE 3
Transporting efficiency to the brain of the transporter peptide

| | Content of the | Injection dose/ | average transport |
|---|---|---|---|
| | transporter peptid | brain tissue | efficiency to |
| Mous | in the brain tissue | weight | the brain |
| sample | (μg) | (% ID/g of brain) | (% ID/g of brain) |
| Brain | 25.5 | 22.09% | 22.79 |
| tissue-1 | | | |
| Brain | 25.9 | 23.48% | |
| tissue-2 | | | |

### Example 5. In Vivo Imaging System (IVIS) Analysis of the Transporter Peptide Conjugated with Antibody

In this Example, the transporter peptide discloed herein is conjugated to an antibody to form a transporter peptide conjugate. The transporter peptide conjugate was then administered to mice, and the distribution of the transporter peptide conjugate in the mice was detected with an in vivo fluorescence imaging system (IVIS). In this example, the transporter peptide conjugate was administered intravenously and orally to analyze the efficacy of different administration routes.

### Materials and Methods

Preparation of Transporter Peptide Conjugate Labeled with Fluorescent Dye

The anti-Her-2 antibody (trastuzumab, Selleckchem, USA) was diluted to a concentration of 1 mg/mL with 100 mM carbonate/bicarbonate buffer. The cross-linking agent (bis(sulfosuccinimidyl) suberate, Thermo Scientific, USA) was dissolved in water to the concentration to 25 mM. One (1) mL of the above-mentioned anti-Her-2 antibody, 11 μl of cross-linking agent solution, and 8.4 μL of the transporter peptide (PT-034, SEQ ID NO: 19, 1 mg/mL) was mixed and incubated at room temperature for 30 minutes for covalent synthesis reaction. The mixture was centrifuged with a 3 KDa filter at 14,000×g for 15 minutes to obtain the transporter peptide conjugate (conjugated to anti-Her-2 antibody). The filtered product was diluted with 50 mM carbonate buffer to the final volume of 1 mL, and 10 μl of 10 mg/mL fluorescent dye (VivoTag 680 XL) was added. The reaction was incubated in the dark at room temperature for 1 hour. The reaction was then centrifuged with a 3 KDa filter at 14,000×g for 15 minutes. The filtered reaction was diluted with 100 mM carbonate buffer to a volume of 0.2 mL to obtain a transporter peptide conjugate labeled with a fluorescent dye (Ab-PT-D).

Preparation of Antibody Labeled with Fluorescent Dye

Ten (10) μl of 10 mg/mL fluorescent dye (VivoTag 680 XL) was added to the anti-Her-2 antibody solution. The mixture was incubated in the dark at room temperature for 1 hour. The mixture was then centrifuged with a 3 KDa filter at 14,000×g for 15 minutes to label the antibody with a fluorescent dye (Ab-D). The filtered mixture was diluted with 50 mM carbonate buffer to a volume of 0.2 mL to obtain the reagent for the negative control group.

### Animal Trail

BALB/c mice (BioLASCO Taiwan Co., Ltd, Taipei, Taiwan) at the age of 7-8 weeks were administered the transporter peptide conjugate labeled with fluorescent dye. Mice were photographed using an optical imaging system (U-OI, MlLabs, Netherlands) at 0.5, 1, 2, 4, 6 and 24 hours after the injection for IVIS imaging analysis, with the excitation wavelength of 631 nm, the emission wavelength of 710 nm, and 10-second exposure time. Fluorescence signals of mouse brains were further quantified.

### Results

### 5.1 Intravenous Injection

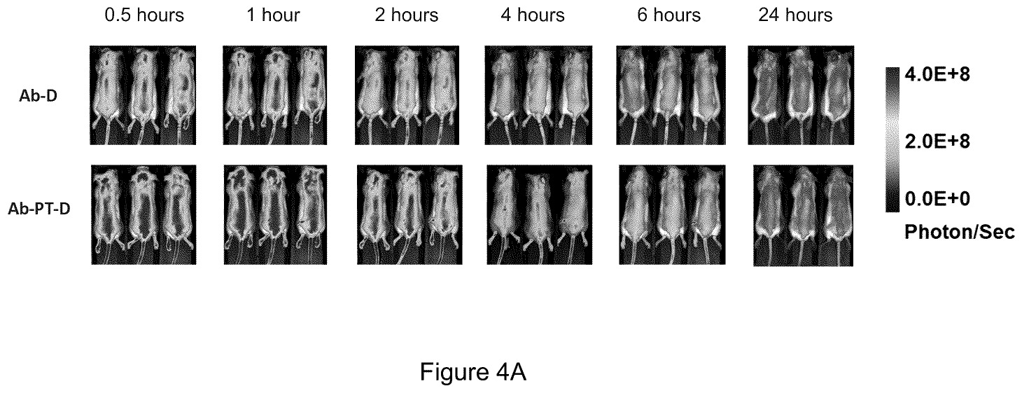

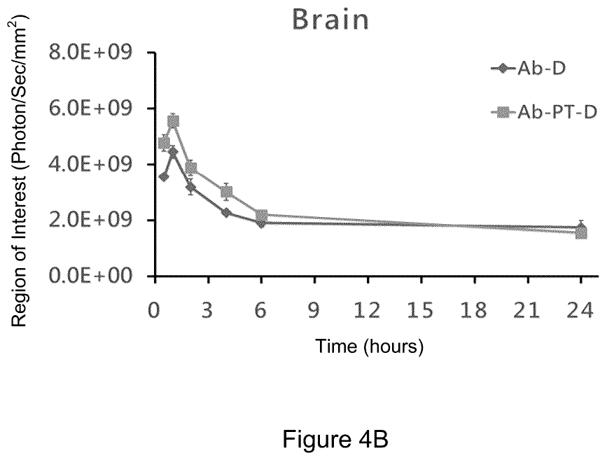

The transporter peptide used in this Example is PT-034 (SEQ ID NO: 19). The mice (n=3) in the control group (Ab-D) were administered 10 mg/kg of the antibody labeled with fluorescent dye via tail vein injection. The mice (n=3) in the test group (Ab-PT-D) were administered 10 mg/kg of the transporter peptide conjugate labeled with fluorescent dye via tail vein injection. As shown in FIG. 4A, at 0.5, 1, 2, 4, and 6 hours after injection, the fluorescent signals were enriched in the brain and spine of the mice in the test group, and the fluorescent signals were stronger than that of the control group. As shown in FIG. 4B, compared with the control group, a higher level of fluorescent signal was observed in the brains of mice in the test group. The results prove that after conjugating the transporter peptide disclosed herein to an antibody, the transporter peptide still has the effect of targeting the brain and spine and can be used as an effective drug delivery system.

### 5.2 Oral Administration

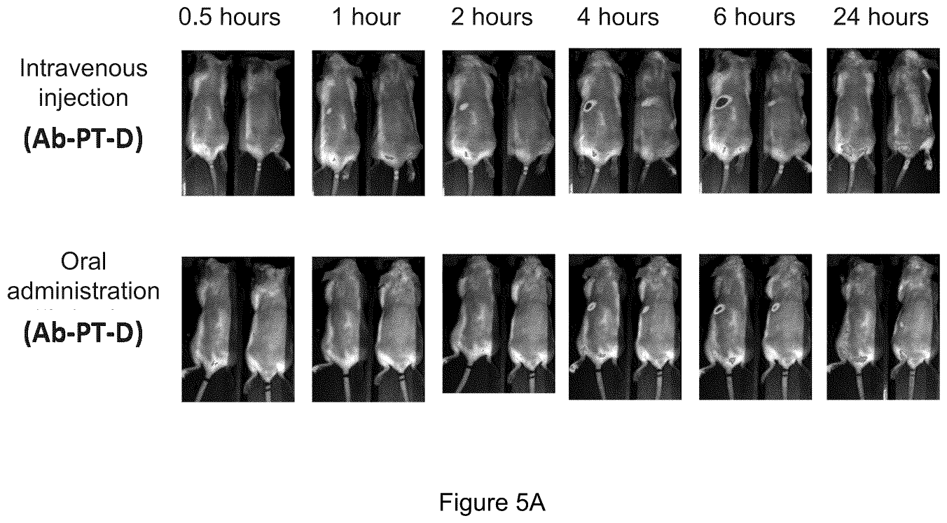

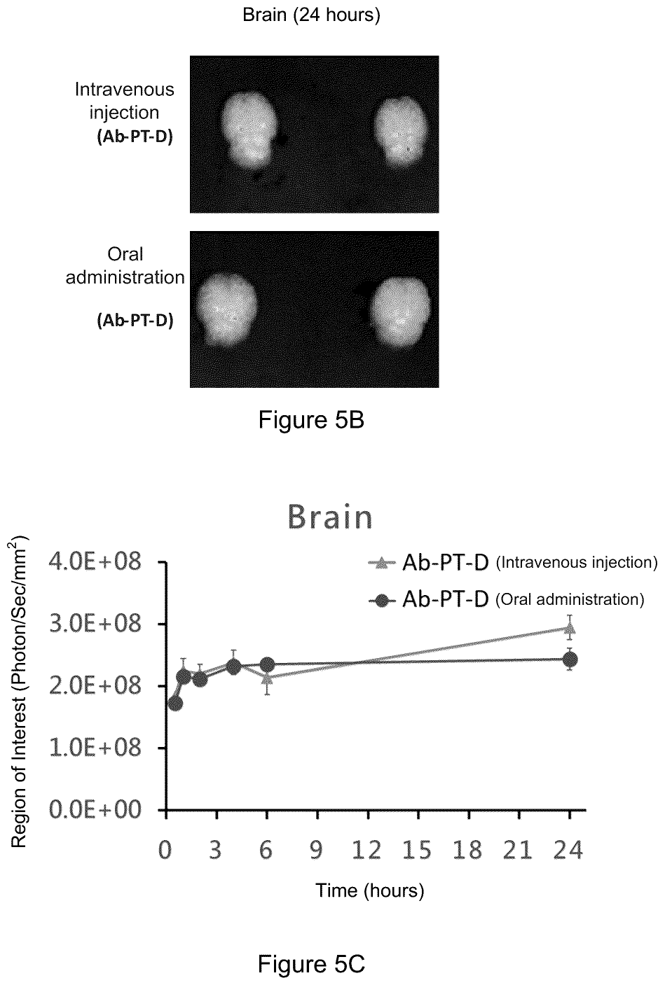

The transporter peptide used in this Example is PT-034 (SEQ ID NO: 19). The mice (n=2) in the intravenous injection (positive control) were administered 0.83 mg/kg of the transporter peptide conjugate labeled with fluorescent dye via tail vein injection. The mice (n=2) in the oral administration group were administered 0.83 mg/kg of the transporter peptide conjugate labeled with fluorescent dye via oral administration. As shown in FIG. 5A, after injection, the fluorescent signals were enriched in the brain and spine of the mice in both groups, indicating that the transporter peptide disclosed herein can be administered orally. As shown in FIG. 5B, obvious fluorescent signals could be detected in the brains of mice administered orally, indicating that the transporter peptide of the present invention can be administered orally. As shown in FIG. 5C, fluorescence signals of mouse brains were further quantified, and the results show that both intravenous injection and oral administration of the transporter peptide conjugate can observe fluorescent signals in mouse brains, proving that the transporter peptide disclosed herein has the effect of targeting the brain via intravenous injection and oral administration and can be used as an effective drug delivery system.

The results indicate that the transporter peptide conjugate of the present invention maintain its efficacy via both intravenous injection or oral administration. Oral administration of the transporter peptide disclosed herein allows the transporter peptide bind to the TfR on the cells of the gastrointestinal mucosal barrier, and the binding of the transporter peptide and the TfR induces transcytosis of the cells, thereby transporting the transporter peptide conjugate across the gastrointestinal mucosal barrier to enter the body and further arrive at the brain. This transporter peptide can also be transported across the blood-brain barrier with the same mechanism.

### Example 6. In Vivo Imaging System (IVIS) Analysis of the Transporter Peptide Conjugated with Lipid Nanoparticles

In this Example, the transporter peptide discloed herein is conjugated to lipid nanoparticles (LNP) to form a transporter peptide conjugate. The transporter peptide conjugate was then administered to mice, and the distribution of the transporter peptide conjugate in the mice was detected with an in vivo fluorescence imaging system (IVIS)

### Materials and Methods

Preparation of Lipid Nanoparticles Labeled with Fluorescent Dye

Two thousand (2000) g of lipid nanoparticles (LNP-102, ABP Biosciences, USA) dissolved in ethanol solution and 200 μg of fluorescent dye (VivoTag 680 XL) dissolved in 50 mM sodium acetate solution were mixed by gentely stirring at room temperature for 30 minutes. The mixture was then dialyzed in PBS in a 2K molecular weight cutoff dialysis box (Slide-A-Lyzer, ThermoFisher Scientific, USA) for 24 hours to obtain lipid nanoparticles labeled with fluorescent dyes (LNP-D).

Preparation of Transporter Peptide Conjugate to Lipid Nanoparticles Labeled with Fluorescent Dye

Zero point one (0.1) mL of 1 mg/mL transporter peptide and 5.8 μl of 20 mg/mL cross-linking agent solution (EZ-Link TFP Ester-PEG4-DBCO, ThermoFisher Scientific Company, United States) were mixed at room temperature for 1 hour. A total of 106 μl of the mixture was added to 475 μl of the above fluorescent dye-labeled lipid nanoparticle solution, and the reaction solution was incubated at room temperature for 1 hour to obtain the transporter peptide conjugate to lipid nanoparticles labeled with fluorescent dye (LNP-PT-D).

### Animal Trail

BALB/c mice (BioLASCO Taiwan Co., Ltd, Taipei, Taiwan) at the age of 7-8 weeks were divided into a control group (n=3) and a test group (n=3). The mice in the control group were administered 10 mg/kg of the lipid nanoparticles labeled with fluorescent dyes (LNP-D). The mice in the test group were administered 10 mg/kg of the transporter peptide conjugate to lipid nanoparticles labeled with fluorescent dye (LNP-PT-D). Mice were photographed using an optical imaging system (U-OI, MlLabs, Netherlands) at 0.5, 1, 2, 4, 6 and 24 hours after the injection for IVIS imaging analysis, with the excitation wavelength of 631 nm, the emission wavelength of 710 nm, and 10-second exposure time. Fluorescence signals of mouse brains were further quantified.

### Results

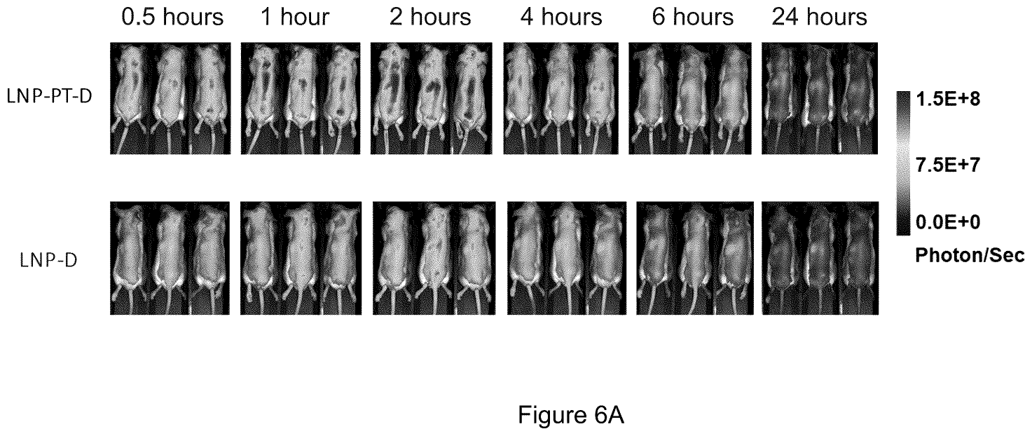

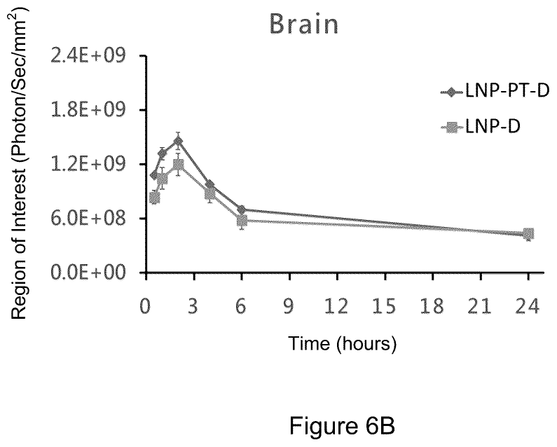

The transporter peptide used in this Example is PT-034 (SEQ ID NO: 19). As shown in FIG. 6A, at 0.5, 1, 2, 4, and 6 hours after injection, the fluorescent signals were enriched in the brain and spine of the mice in the test group, and the fluorescent signals were stronger than that of the control group. As shown in FIG. 6B, compared with the control group, a higher level of fluorescent signal was observed in the brains of mice in the test group. The results prove that after conjugating the transporter peptide disclosed herein to lipid nanoparticles, the transporter peptide still has the effect of targeting the brain and spine and can be used as an effective drug delivery system.

To sum up, the Examples disclosed herein prove that the tranporter peptides of the present invention have binding affinity to TfR. The binding of the tranporter peptides to TfRs on cells of a tissue barrier induces transcytosis of the cells, thereby transporting the transporter peptide conjugate across the gastrointestinal mucosal barrier to enter the body and further arrive at the brain. The data in the Examples show that each site of the transporter peptides of the present invention (SEQ ID NO: 1) can be substituted by several specific amino acids without loss of binding affinity to TfR. In addition, the transporter peptides of the present invention can be conjugated to an effector agent to form a transporter peptides conjugate. The data in the Examples show that the transporter peptides conjugates maintain their binding affinitys to TfR. The transporter peptides conjugates can deliver the effector agent across a tissue barrier and effectively overcome the problem of extremely low drug passing rate across the blood-brain barrier. Therefore, the transporter peptides of the present invention can be broadly used for the treatment and/or prevention of CNS diseases, and is also beneficial to the development of related clinical medical application.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

1. A transporter peptide, consisting of a sequence of SEQ ID NO: 1, and the transporter peptide being able to bind to a transferrin receptor (TfR).

2. The transporter peptide of claim 1, wherein the sequence of the transporter peptide is selected from the group consisting of SEQ ID NO: 2 to SEQ ID NO: 30 and variant sequences at least 70% homologous to one of SEQ ID NO: 2 to SEQ ID NO: 30 and being able to bind to the transferrin receptor.

3. A nucleic acid encoding the transporter peptide of claim 1.

4. The nucleic acid of claim 3, wherein the nucleic acid is deoxyribonucleic acid (DNA) or ribonucleic acid (RNA).

5. A vector, comprising a nucleic acid encoding the transporter peptide of claim 1.

6. The vector of claim 5, wherein the vector is an adeno-associated virus (AAV) vector.

7. The vector of claim 5, further comprising a nucleic acid encoding an effector agent.

8. The vector of claim 7, wherein the effector agent is at least one selected from the group consisting of peptides, proteins, antibodies, viral particles, liposomes, endosomes, exosomes, ligands, eukaryotic cells, prokaryotic cells, and microspheres.

9. A recombinant transporter peptide conjugate, which is expressed by a vector comprising a nucleic acid encoding the transporter peptide of claim 1 and a nucleic acid encoding an effector agent.

10. A recombinant host cell, comprising at least one component selected from the group consisting of
the transporter peptide of claim 1;
a nucleic acid encoding the transporter peptide of claim 1; and
a vector comprising a nucleic acid encoding the transporter peptide of claim 1.

11. A transporter peptide conjugate, consisting of the transporter peptide of claim 1 and an effector agent, and the effector agent being covalently or non-covalently conjugated to the transporter peptide.

12. transporter peptide conjugate of claim 11, wherein the effector agent is at least one selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

13. A composition, comprising at least one selected from the group consisting of
the transporter peptide of claim 1;
a recombinant transporter peptide conjugate expressed by a vector comprising a nucleic acid encoding the transporter peptide of claim 1 and a nucleic acid encoding an effector agent; and
a transporter peptide conjugate consisting of the transporter peptide of claim 1 and an effector agent covalently or non-covalently conjugated to the transporter peptide.

14. The composition of claim 13, further comprising a pharmaceutically acceptable carrier.

15. A method of transporting the composition of claim 13 to a target, the method comprising binding the transporter peptide, the transporter peptide of the recombinant transporter peptide conjugate, or the transporter peptide of the transporter peptide conjugate to a transferrin receptor.

16. The method of claim 15, wherein the composition has to be transported across a tissue barrier to arrive at the target, and the transferrin receptor is located on a cell of the tissue barrier.

17. The method of claim 16, wherein the tissue barrier is one of a blood-brain barrier (BBB), a mucosal barrier, and a gastrointestinal barrier.

18. The method of claim 17, wherein binding of the transporter peptide of the composition, the transporter peptide of the transporter peptide conjugate of the composition, or the transporter peptide of the recombinant transporter peptide conjugate of the composition to the transferrin receptor induces transcytosis of the cells, thereby transporting the composition across the tissue barrier to arrive at the target.

19. The method of claim 15, wherein the target is a cell expressing the transferrin receptor, and the transferrin receptor is located on the target.

20. The method of claim 15, wherein the target is in vivo or in vitro.

21. A method of transporting an effector agent across a tissue barrier of a subject, comprising administering to the subject a recombinant transporter peptide conjugate expressed by a vector comprising a nucleic acid encoding the transporter peptide of claim 1 and a nucleic acid encoding an effector agent; or
a transporter peptide conjugate consisting of the transporter peptide of claim 1 and an effector agent covalently or non-covalently conjugated to the transporter peptide.

22. The method of claim 21, wherein the tissue barrier is one of a blood-brain barrier (BBB), a mucosal barrier, and a gastrointestinal barrier.

23. The method of claim 22, wherein cells of the tissue barrier express transferrin receptors.

24. The method of claim 23, wherein the transporter peptide binds to the transferrin receptor, and the binding of the transporter peptide to the transferrin receptor induces transcytosis of the cells, thereby transporting the transporter peptide and the effector agent across the tissue barrier.

25. The method of claim 21, wherein the recombinant transporter peptide conjugate or the transporter peptide conjugate is administered to the subject by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

26. The method of claim 21, wherein the effector agent in the recombinant transporter peptide conjugate is at least one selected from the group consisting of peptides, proteins, antibodies, viral particles, liposomes, endosomes, exosomes, ligands, eukaryotic cells, prokaryotic cells, and microspheres.

27. The method of claim 21, wherein the effector agent consisted in the transporter peptide conjugate is at least one selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

28. The method of claim 21, wherein the subject is a mammal.

29. The method of claim 28, wherein the subject is a rodent or a human.

30. A method of treating or preventing a central nervous system (CNS) disease, comprising administering a subject in need thereof a pharmaceutically effective amount of
a recombinant transporter peptide conjugate expressed by a vector comprising a nucleic acid encoding the transporter peptide of claim 1 and a nucleic acid encoding an effector agent; or
a transporter peptide conjugate consisting of the transporter peptide of claim 1 and an effector agent covalently or non-covalently conjugated to the transporter peptide.

31. The method of claim 30, wherein the effector agent in the recombinant transporter peptide conjugate or consisted in the transporter peptide conjugate is a therapeutic agent of the CNS disease.

32. The method of claim 30, wherein the transporter peptide of the recombinant transporter peptide conjugate or the transporter peptide of the transporter peptide conjugate binds to a transferrin receptor on a cell of a tissue barrier of the subject, and the binding of the transporter peptide to the transferrin receptor induces transcytosis of the cell, thereby transporting the transporter peptide and the therapeutic agent of the CNS disease across the tissue barrier.

33. The method of claim 32, wherein the tissue barrier is one of a blood-brain barrier (BBB), a mucosal barrier, and a gastrointestinal barrier.

34. The method of claim 30, wherein the subject is a mammal.

35. The method of claim 34, wherein the subject is a rodent or a human.

36. The method of claim 30, wherein the recombinant transporter peptide conjugate or the transporter peptide conjugate is administered to the subject in need thereof by one of intradermal delivery, intramuscular delivery, subcutaneous delivery, intravenous delivery, intra-atrial delivery, intra-articular delivery, intraperitoneal delivery, parenteral delivery, oral delivery, rectal delivery, intranasal delivery, intrapulmonary delivery, and transdermal delivery.

37. The method of claim 30, wherein the CNS disease is one of Alzheimer's disease (AD), Parkinson's disease (PD), cerebrovascular accidents (CVA), ascular-related dementia, Creutzfeldt-Jakob disease (CJD), bovine spongiform encephalopathy (BSE), Traumatic Brain Injury (TBI), multiple sclerosis (MS), amyotrophic lateral sclerosis (ALS), Huntington's chorea, and spinal muscular atrophy (SMA).

38. The recombinant transporter peptide conjugate of claim 9, wherein the effector agent is at least one selected from the group consisting of peptides, proteins, antibodies, viral particles, liposomes, endosomes, exosomes, ligands, eukaryotic cells, prokaryotic cells, and microspheres.

39. The composition of claim 13, wherein the effector agent in the recombinant transporter peptide conjugate is at least one selected from the group consisting of peptides, proteins, antibodies, viral particles, liposomes, endosomes, exosomes, ligands, eukaryotic cells, prokaryotic cells, and microspheres.

40. The composition of claim 13, wherein the effector agent consisted in the transporter peptide conjugate is at least one selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.

41. The method of claim 30, wherein the effector agent in the recombinant transporter peptide conjugate is at least one selected from the group consisting of peptides, proteins, antibodies, viral particles, liposomes, endosomes, exosomes, ligands, eukaryotic cells, prokaryotic cells, and microspheres.

42. The method of claim 30, wherein the effector agent consisted in the transporter peptide conjugate is at least one selected from the group consisting of siRNA, shRNA, microRNA, double-stranded RNA, single-stranded RNA, DNA, oligonucleotides, aptamers, genes, peptides, proteins, antibodies, small chemical molecules, large chemical molecules, viral particles, liposomes, endosomes, exosomes, nanoparticles, lipid nanoparticle, dendrimers, ligands, eukaryotic cells, prokaryotic cells, microspheres, nanogels, and bionanocapsules.


SEQUENCE LISTING (30 sequences)

SEQ ID NO: 1 - protein, 10 aa, synthetic construct
  VARIANT 1: note = D, E, N, Q, C, S, T or Y
  VARIANT 2: note = G, A, I, L, V, P, F, W or M
  VARIANT 3: note = H, R, K, D, E, A, I, L, V, P, F, W, M, G, N, Q, C, S, T or Y
  VARIANT 4: note = D, E, G, A, I, L, V, P, F, W or M
  VARIANT 5: note = G, A, I, L, V, P, F, W or M
  VARIANT 6: note = H, R, K, D, E, A, I, L, V, P, F, W, M, G, N, Q, C, S, T or Y
  VARIANT 7: note = H, R or K
  VARIANT 8: note = H, R, K, D or E
  VARIANT 9: note = G, A, I, L, V, P, F, W, M, N, Q, C, S, T or Y
  VARIANT 10: note = D, E, N, Q, C, S, T or Y
XIXVLXKXXX

SEQ ID NO: 2 - protein, 10 aa, synthetic construct
DISVLMKEYD

SEQ ID NO: 3 - protein, 10 aa, synthetic construct
DIDVLMKEYD

SEQ ID NO: 4 - protein, 10 aa, synthetic construct
DIHVLMKEYD

SEQ ID NO: 5 - protein, 10 aa, synthetic construct
DILVLMKEYD

SEQ ID NO: 6 - protein, 10 aa, synthetic construct
DIPVLMKEYD

SEQ ID NO: 7 - protein, 10 aa, synthetic construct
DITVLMKEYD

SEQ ID NO: 8 - protein, 10 aa, synthetic construct
DISLLMKEYD

SEQ ID NO: 9 - protein, 10 aa, synthetic construct
DISVIMKEYD

SEQ ID NO: 10 - protein, 10 aa, synthetic construct
DISVVMKEYD

SEQ ID NO: 11 - protein, 10 aa, synthetic construct
DISVLDKEYD

SEQ ID NO: 12 - protein, 10 aa, synthetic construct
DISVLNKEYD

SEQ ID NO: 13 - protein, 10 aa, synthetic construct
DISVLCKEYD

SEQ ID NO: 14 - protein, 10 aa, synthetic construct
DISVLKKEYD

SEQ ID NO: 15 - protein, 10 aa, synthetic construct
DISVLEKEYD

SEQ ID NO: 16 - protein, 10 aa, synthetic construct
DISVLQKEYD

SEQ ID NO: 17 - protein, 10 aa, synthetic construct
DISVLMKEYN

SEQ ID NO: 18 - protein, 10 aa, synthetic construct
DISVLMKEYE

SEQ ID NO: 19 - protein, 10 aa, synthetic construct
DISVLMKEYQ

SEQ ID NO: 20 - protein, 10 aa, synthetic construct
DISVLSKEYD

SEQ ID NO: 21 - protein, 10 aa, synthetic construct
DISVLMKDYD

SEQ ID NO: 22 - protein, 10 aa, synthetic construct
DISVLMKEFD

SEQ ID NO: 23 - protein, 10 aa, synthetic construct
DISVLMKESD

SEQ ID NO: 24 - protein, 10 aa, synthetic construct
DISVLMKETD

SEQ ID NO: 25 - protein, 10 aa, synthetic construct
NISVLMKEYD

SEQ ID NO: 26 - protein, 10 aa, synthetic construct
DISELMKEYD

SEQ ID NO: 27 - protein, 10 aa, synthetic construct
DISVLLKEYD

SEQ ID NO: 28 - protein, 10 aa, synthetic construct
DISVLMKHYD

SEQ ID NO: 29 - protein, 10 aa, synthetic construct
DITVLCKEYN

SEQ ID NO: 30 - protein, 10 aa, synthetic construct
DIHVLDKEYD